(19) United States
(12) Patent Application Publication
(10) Pub. No.: US 2010/0317949 A1
(43) Pub. Date: Dec. 16, 2010

(54) OPTICAL COUPLER FOR NON-INVASIVE SPECTROPHOTOMETRIC PATIENT MONITORING

(75) Inventors: Eric V. CHAMNESS, Mountain View, CA (US); Wei Zhang, Los Altos, CA (US)

Correspondence Address: COOPER & DUNHAM, LLP, 30 Rockefeller Plaza, 20th Floor, NEW YORK, NY, 10112, US

(73) Assignee: 02 MEDTECH, INC., Los Altos, CA (US)

(21) Appl. No.: 12/483,610

(22) Filed: Jun. 12, 2009

(51) Int. Cl. A61B 5/1455

(52) U.S. Cl. 600/344

(57) ABSTRACT

Flexible, low-cost, physically robust optical coupling patches for use in spectrophotometric patient monitoring, and methods of fabrication thereof, are described. The optical coupling patch comprises a flexible base layer having a skin-contacting surface and a first aperture formed therethrough that establishes an optical interface with a skin surface when the base layer is placed against the skin surface. The optical coupling patch further comprises an elastomeric waveguiding member laterally disposed on a surface of the base layer opposite the skin-contacting surface. The optical coupling patch guides optical radiation between a laterally propagating state at a first location laterally distal from the first aperture and a generally vertically propagating state at the first aperture.

### FIELD

This patent specification relates to non-invasive spectrophotometric patient monitoring in which optical radiation, such as near-infrared optical radiation, is directed onto a skin surface of the patient, migrates through at least a portion of a tissue sample underlying the skin surface, and then is measured as it emanates outwardly again from the skin surface. More particularly, this patent specification relates to an optical coupler for directing the optical radiation onto the skin surface and collecting for measurement the resultant outwardly emanating optical radiation.

### BACKGROUND

Spectrophotometric systems based on visible and/or near infrared (NIR) optical radiation for achieving various non-invasive physiological measurements, such as transcranial measurements of oxygenated hemoglobin (HbO) and deoxygenated hemoglobin (Hb) concentrations, have been in various stages of proposal and development for an appreciable number of years. Examples include continuous wave (CWS) spectrophotometric systems as discussed in WO1992/20273A2 and WO1996/16592A1, phase modulation (PMS) spectrophotometric systems as discussed in U.S. Pat. No. 4,972,331, U.S. Pat. No. 5,187,672, and WO1994/21173A1, time resolved (TRS) spectrophotometric systems as discussed in U.S. Pat. No. 5,119,815, U.S. Pat. No. 5,386,827, and WO1994/22361A1, and phased array spectrophotometric systems as discussed in WO1993/25145A1, each of these disclosures being incorporated by reference herein. It is to be appreciated that while an optical coupler according to one or more of the preferred embodiments described infra is particularly suitable for use in a non-invasive cerebral oxygenation monitoring context, the scope of the present teachings is not so limited, with one or more of the described optical couplers being readily adapted for use on other parts of the anatomy, such as the neck, the abdomen, the arms, and the legs.

FIG. 1A illustrates one prior art optical coupling configuration in which source radiation is guided from an external optical source into a tissue sample 4 by a source fiber optic cable 1, and in which detected radiation is guided to one or more external measurement devices by return fiber optic cables 2, wherein the source and return fiber optic cables are cemented into a rigid holder 3 for direct perpendicular abutment against the skin surface. Although perhaps suitable for laboratory experiments, the configuration of FIG. 1A becomes impractical in real-world clinical settings in which patients, who may be lying, sitting, or standing in various positions, require monitoring over a substantial period of time. In such cases, unfavorable leverages make it difficult to maintain the holder 3 in a secured position relative to the skin surface over a period of time in a reasonably comfortable manner as would be needed for consistency of measurement and prevention of ambient light intrusion.

FIG. 1B illustrates a different prior art approach to optical coupling as discussed in U.S. Pat. No. 5,584,296, in which both the optical sources and the optical detectors are integrated into a deformable coupling patch. External cabling requirements are made easier since only electrical power and electrical information signals need be carried to and from the device, and in view of its relative flatness and conformability, the device can be secured to the patient in a more stable and comfortable manner. One particular advantage is the ability for the cable leads to lie flat against the body, so that the both the cable leads and the patch can be readily secured in place. However, the semiconductor photodiodes needed for the on-patch optical detection are substantially less sensitive than off-patch detection solutions such as photomultiplier tubes (PMTs), resulting in lower signal-to-noise performance than if PMTs were used. Likewise, due to size and heat restrictions, the on-patch optical sources are of lesser precision and power than can be supplied using larger and more powerful off-patch sources. Electromagnetic shielding problems due to the presence of radio frequency (RF) radiation also become problematic. Moreover, the complexity brought about by the various electrical connections and RF shielding hardware reduce the mechanical flexibility and robustness of the device, such that it needs to be treated rather tenderly to reduce the risk of malfunction. Finally, the complexity of the device also increases the fabrication cost such that disposability is not a realistic option, thus bringing about the need for costly decontamination procedures between patients and/or the need for awkward, performance-reducing prophylactic sheathing measures.

FIG. 1C illustrates another prior art approach to optical coupling as discussed in U.S. Pat. No. 7,313,427, in which an optical detector is integrated into the deformable coupling patch, but in which the source optical signal is provided externally. The optical signal is guided laterally over a fiber optic cable from an edge of the coupling patch to the location at which light insertion is desired, and then is redirected vertically by use of a prism into the skin at that location. Although source precision and power issues may be improved over the configuration of FIG. 1B, supra, many of the other disadvantages remain, such as lower signal-to-noise ratios associated with the photodiode detectors, electromagnetic shielding problems, device cost, device complexity, and reduced mechanical flexibility and robustness.

FIG. 1D illustrates another prior art approach to optical coupling as discussed in U.S. Pat. No. 4,510,938, in which both the optical sources and the optical detectors are provided externally. A first optical fiber bundle is used to transfer the source radiation laterally across a coupling structure to the desired location of light insertion, at which point the first optical fiber bundle is bent at a right angle to direct the source radiation downward onto the skin surface. A second optical fiber bundle is similarly configured for receiving the outwardly emanating radiation and transferring that radiation to an external optical detector. Disadvantageously, a substantial amount of device height in a direction outward from the skin surface (i.e., a substantial amount of overall device thickness) is needed to accommodate the bending of the optical fiber bundles. The larger size brings about positional stability issues, and the presence of the optical fiber bundles contributes to reduced device flexibility/conformability as well as physical robustness issues.

FIG. 1E illustrates another prior art approach to optical coupling as discussed in U.S. Pat. No. 6,556,851, in which both the optical sources and the optical detectors are provided externally, and in which prisms are used to avoid the need for right-angle bending of the optical fiber cables. Although the overall device can be flatter than that of FIG. 1D, supra, the optical fiber cables can limit flexibility and conformability, as well as bring about problems with device robustness against rough handling. By way of example, a substantial degree of device bending can damage the optical fiber cables and/or disturb their physical relationship to the prisms at one or more failure points, causing a reduction in performance and/or device failure.

It would be desirable to provide an optical coupling device for use in non-invasive spectrophotometric patient monitoring that provides an advantageous combination of physical robustness, relatively low fabrication cost, minimal profile thickness, disposability, and durability, while also providing for effective optical coupling with good signal to noise performance. Each of the above-described prior art optical coupling configurations is believed to bring about one or more disadvantages and/or to contain one or more shortcomings that is avoided by one or more devices or techniques according to one or more of the preferred embodiments described hereinbelow. Other issues arise as would be apparent to one skilled in the art upon reading the present disclosure.

### SUMMARY

According to one preferred embodiment, a flexible, low-cost, physically robust optical coupling patch is provided for use in spectrophotometric patient monitoring. The optical coupling patch comprises a flexible base layer having a skin-contacting surface and a first aperture formed therethrough, the flexible base layer comprising a first elastomeric material having a first refractive index, the first aperture establishing an optical interface with a skin surface when the flexible base layer is placed against the skin surface. The optical coupling patch further comprises an elastomeric waveguiding member laterally disposed on a surface of the flexible base layer opposite the skin-contacting surface. The elastomeric waveguiding member comprises a second elastomeric material having a second refractive index greater than the first refractive index and is configured to guide optical radiation between (i) a laterally propagating state at a first location laterally distal from the first aperture, and (ii) a generally vertically propagating state at the first aperture. The elastomeric waveguiding member includes a substantially planar reflecting surface shaped integrally thereinto near the first aperture. The reflecting surface is oriented at an angle that causes reflective redirection of the optical radiation between the laterally propagating state and the vertically propagating state. The optical coupling patch further comprises a flexible cladding material having a third refractive index less than the second refractive index. The flexible cladding material selectively covers the elastomeric waveguiding member such that a cavity is formed directly adjacent the integrally formed reflecting surface of the elastomeric waveguiding member, wherein the cavity is occupied by either air or a low-index material having a fourth refractive index less than the third refractive index, whereby the reflective redirection of the optical radiation is facilitated.

Also provided according to a preferred embodiment is an optical coupling patch for use in spectrophotometric patient monitoring having a bottom surface for contacting a skin surface of a patient and a side edge including first and second end facets. The optical coupling patch is operable to guide source radiation received at the first end facet to a downward facing first aperture formed in the bottom surface for downward introduction into the patient. The coupling patch is further operable to receive radiation emanating upwardly from the patient at a second aperture formed in the bottom surface and to guide the received radiation from the second aperture to the second end facet. The optical coupling patch comprises a flexible base layer, first and second elastomeric waveguiding members disposed on the base layer and extending from the first and second end facets, respectively, to the first and second apertures, respectively. The optical coupling patch further comprises a flexible first cladding layer disposed on the base layer and extending alongside the first and second elastomeric waveguiding members, and a flexible second cladding layer disposed atop the first cladding layer and the first and second elastomeric waveguiding members. The base layer, the first cladding layer, and the second cladding layer each have an index of refraction less than that of either of the first and second elastomeric waveguiding members. The first and second elastomeric waveguiding members each include a substantially planar surface shaped integrally thereinto near its respective aperture that is oriented at an angle between about 35 and 55 degrees relative thereto, whereby the source radiation propagating laterally in the first elastomeric waveguiding member is reflectively redirected downward toward the first aperture, and whereby the upwardly emanating radiation received at the second aperture is reflectively redirected to propagate laterally in the second elastomeric waveguiding member toward the second end facet.

Also provided according to another preferred embodiment is a method for fabricating a flexible, slab-like optical coupling patch for use in spectrophotometric patient monitoring, the optical coupling patch having a bottom surface for contacting a skin surface of a patient, and a side edge. The method comprises providing a flexible base layer comprising an elastomeric material, the base layer extending to the side edge and having a lower surface corresponding to the bottom surface of the optical coupling patch and an upper surface opposite the lower surface, the base layer having an opening extending through the lower and upper surfaces thereof. The method further comprises forming an elastomeric waveguiding member on the upper surface of the base layer extending laterally thereacross between the side edge and the opening, the elastomeric waveguiding member comprising a first end facet facing in a lateral direction at the side edge, a second end facet facing downwardly into the first opening, and a substantially planar surface integrally formed into the first elastomeric waveguiding member by virtue of its outer shape at a location directly above the second end facet, the substantially planar surface being oriented at an angle between about 35 and 55 degrees relative to the second end facet. The method further comprises forming at least one flexible cladding layer that covers the base layer and the elastomeric waveguiding member.

Also provided according to another preferred embodiment is a flexible, slab-like optical coupling patch for use in spectrophotometric patient monitoring. The optical coupling patch has a bottom surface for contacting a skin surface of a patient and a downward facing first aperture formed in the bottom surface. The optical coupling patch is operable to guide optical radiation between (i) a laterally propagating state at a first location laterally distal from the first aperture, and (ii) a generally vertically propagating state at the first aperture. The optical coupling patch comprises a flexible base layer including the skin-contacting surface and a first opening formed therethrough that establishes the first aperture. The first aperture establishes an optical interface with the skin surface when the flexible base layer is placed thereagainst. The optical coupling patch further comprises a light deflecting member disposed above the first opening and configured to deflect optical radiation between a generally vertically propagating state thereat and a laterally propagating state thereat. The optical coupling patch further comprises an elastomeric waveguiding member disposed on a surface of the flexible base layer opposite the skin-contacting surface and extending laterally across the optical coupling patch from the light deflecting member to the first location laterally distal from the first aperture.

### BRIEF DESCRIPTION OF THE DRAWINGS

FIGS. 1A-1E each illustrate an optical coupler according to the prior art;

FIG. 2 illustrates a non-invasive spectrophotometric patient monitoring system including an optical coupling assembly according to a preferred embodiment;

FIGS. 3A-3B illustrate perspective views of an optical coupling patch according to a preferred embodiment;

FIG. 4A illustrates a bottom view of an optical coupling patch according to a preferred embodiment;

FIGS. 4B-4D illustrate side cut-away views of the optical coupling patch of FIG. 4A;

FIGS. 5A-5B illustrate exploded perspective views of the optical coupling patch of FIG. 4A;

FIG. 6 illustrates a side cut-away view of an optical coupling assembly according to a preferred embodiment;

FIGS. 7A-7B illustrate perspective views of an optical coupling patch according to a preferred embodiment;

FIG. 8 illustrates a bottom view of an optical coupling patch according to a preferred embodiment;

FIG. 9 illustrates an exploded perspective views of the optical coupling patch of FIG. 8;

FIG. 10 illustrates a perspective view of an optical coupling patch according to a preferred embodiment;

FIG. 11 illustrates a side cut-away view of an optical coupling patch according to a preferred embodiment;

FIG. 12 illustrates a side cut-away view of an optical coupling patch according to a preferred embodiment;

FIG. 13 illustrates a side cut-away view of an optical coupling patch according to a preferred embodiment;

FIG. 14 illustrates a side cut-away view of an optical coupling patch according to a preferred embodiment; and

FIG. 15 illustrates a side cut-away view of an optical coupling patch according to a preferred embodiment.

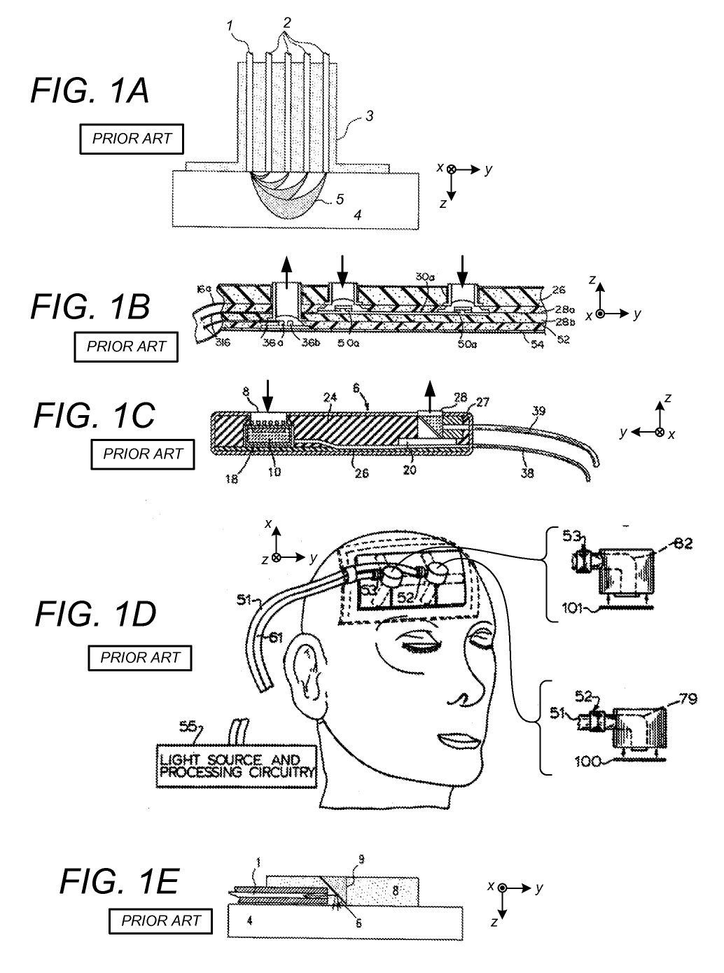

### DETAILED DESCRIPTION

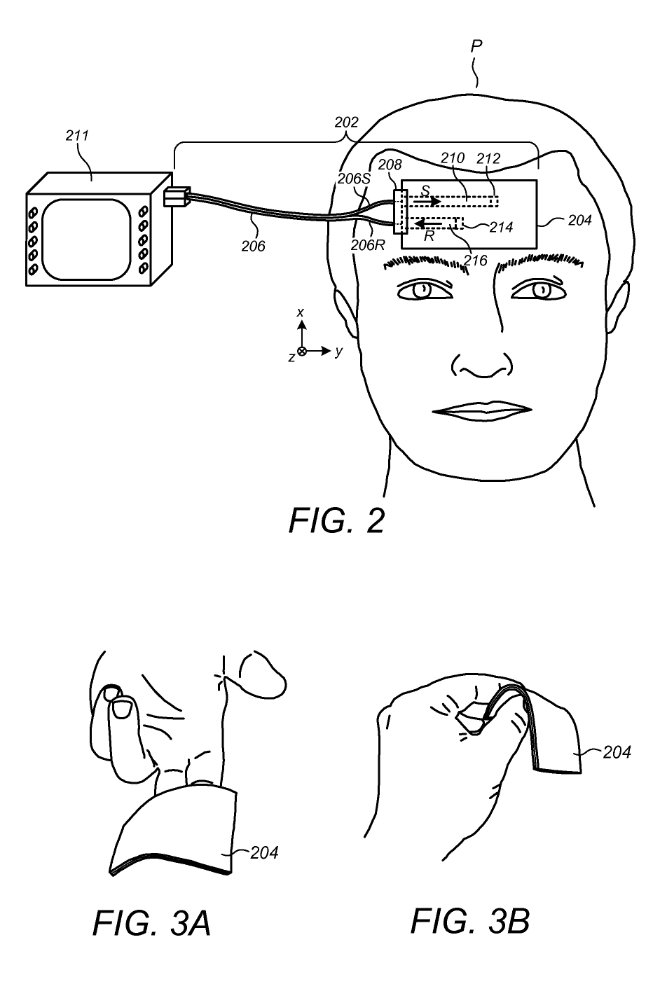

FIG. 2 illustrates a spectrophotometric patient monitoring system including a console unit 211 and an optical coupling apparatus 202 according to a preferred embodiment. The optical coupling apparatus 202 is entirely passive, containing no optical signal generation devices or electrooptical detection devices, but rather is configured to transfer source optical radiation from the console unit 211 into a skin surface of a patient P, and to receive and transfer optical radiation emanating outwardly from the skin surface back to the console unit 211 for measurement. Optical coupling apparatus 202 comprises a fiber optic cable assembly 206 including a source fiber optic cable 206S and a return fiber optic cable 206R, each preferably containing a bundle of optical fibers. The source and return fiber optic cables 206S and 206R are coupled at one end to the console unit 211 and at the other end to an optical coupling patch 204 via an edge adapter 208.

The console unit 211 includes one or more optical sources, such as a laser source, and one or more optical detectors, such as a photomultiplier tube (PMT), along with associated control, processing, and display circuitry as may be used with any of a variety of spectrophotometric techniques. One wavelength range for which the optical coupling apparatus 202 is suitable is the 500 nm-1000 nm range. The optical coupling apparatus 202 is particularly suitable for use with optical radiation in the range of 690 nm-830 nm, although the scope of the preferred embodiments is not so limited.

Optical coupling patch 204 comprises a flexible, thin, low-profile, generally slab-like body designed to be easily brought into contact with the skin surface of the patient and maintained thereagainst over a relatively long time period while also being comfortable. Any of a variety of methods, or combination of methods, for maintaining the optical coupling patch 204 in contact with the skin surface are within the scope of the preferred embodiments including, but not limited to: directly adhering a bottom surface of the optical coupling patch 204 to the skin using an adhesive; adhering the optical coupling patch 204 to the skin around a periphery thereof using an oversized adhesive patch; and using various elastic wrap or ACE® bandaging configurations. In one preferred embodiment that is particularly applicable to cerebral spectrophotometric monitoring, the optical coupling patch 204 can be affixed on the inside of an headband assembly, a wearable hat assembly, or helmet assembly that is worn by the patient during the monitoring session.

As used herein with respect to optical coupling patch 204, the term lateral direction refers to a direction generally parallel to or along the patient's skin surface when the optical coupling patch 204 is positioned thereagainst, while the term vertical direction refers to a direction generally normal to the skin surface when the optical coupling patch 204 is positioned thereagainst (i.e., an inward/outward direction with respect to the skin surface). Thus, it is to be appreciated that the terms “lateral” and “vertical” as used herein with respect to optical coupling patch 204 do not imply any particular direction with respect to gravity or other fixed frame of reference in the surrounding clinical environment. It is to be further appreciated that the term “lateral” as used herein with respect to optical coupling patch 204 does not imply restriction to a single geometric plane, which is particularly relevant for cases in which the optical coupling patch 204 is applied for monitoring of the neck, arms, legs, feet, or fingers, or when the optical coupling patch 204 is only partially supported or lying on a non-planar surface.

Integrally formed into optical coupling patch 204 is a source elastomeric waveguiding member 210 configured and dimensioned to transfer source optical radiation laterally from an edge of the optical coupling patch 204 to an emitting aperture 212 that faces downwardly into the skin surface. Also integrally formed into optical coupling patch 204 is a detection elastomeric waveguiding member 216 configured and dimensioned to receive radiation emanating upwardly at a detection aperture 214 and to transfer that radiation laterally to the edge of the optical coupling patch 204. Although the source elastomeric waveguiding member 210 and detection elastomeric waveguiding member 216 preferably terminate near each other along a common side of the optical coupling patch 204, thereby simplifying optical fiber cabling requirements, the scope of the present teachings is not so limited and includes configurations in which the source elastomeric waveguiding member 210 and detection elastomeric waveguiding member 216 terminate along different sides of the optical coupling patch 204.

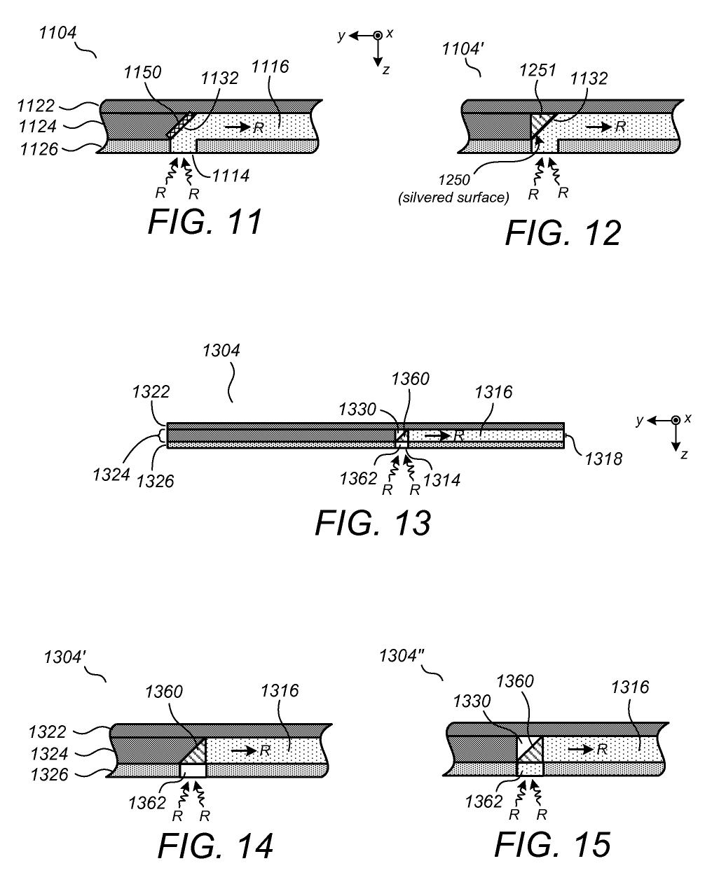

FIGS. 3A and 3B illustrate perspective views of the optical coupling patch 204 as held in a hand, with edge adapter 208 and fiber optic cable assembly 206 omitted. In one exemplary preferred embodiment, the optical coupling patch 204 has lateral dimensions of about 3 inches (7.62 cm) by 1.5 inches (3.81 cm), and a thickness of about 0.15 inches (3.8 mm). In one preferred embodiment, the optical coupling patch 204 is entirely elastomeric in construction, with no optical fiber bundles and no rigid components contained therein, for providing an advantageous combination of conformability, durability, and low fabrication cost. In alternative preferred embodiments to be described further infra (see FIGS. 11-15) a rigid reflective optical component, such as a prism or a planar mirror element, can be positioned near each elastomeric waveguiding member, but that element is sufficiently small such that the overall flexible, bendable, and “floppy” physical character of the optical coupling patch is not substantially affected.

In accordance with a preferred embodiment, the optical coupling patch 204 comprises a multilayer structure in which each layer is formed from a thermally curable polysiloxane elastomer having a Shore OO durometer hardness in the range of 25 to 95. In another preferred embodiment, the polysiloxane elastomer exhibits a Shore A durometer hardness in the range of 20 to 60. In other preferred embodiments, the polysiloxane elastomer exhibits a Shore A durometer hardness in the range of 10 to 90. In addition to flexibility, durability, and low cost, the class of preferred polysiloxane elastomers further exhibits chemical inertness, water repellency, electrical insulation properties, and biocompatibility. Other classes of elastomeric materials that may be usable in conjunction with one or more of the preferred embodiments include certain flexible polybutadienes, epoxy resins, and polyurethanes, and more generally any elastomeric material known or hereinafter developed that possesses equivalent optical and mechanical properties to the described polysiloxane elastomers while being sufficiently safe for placement on human skin.

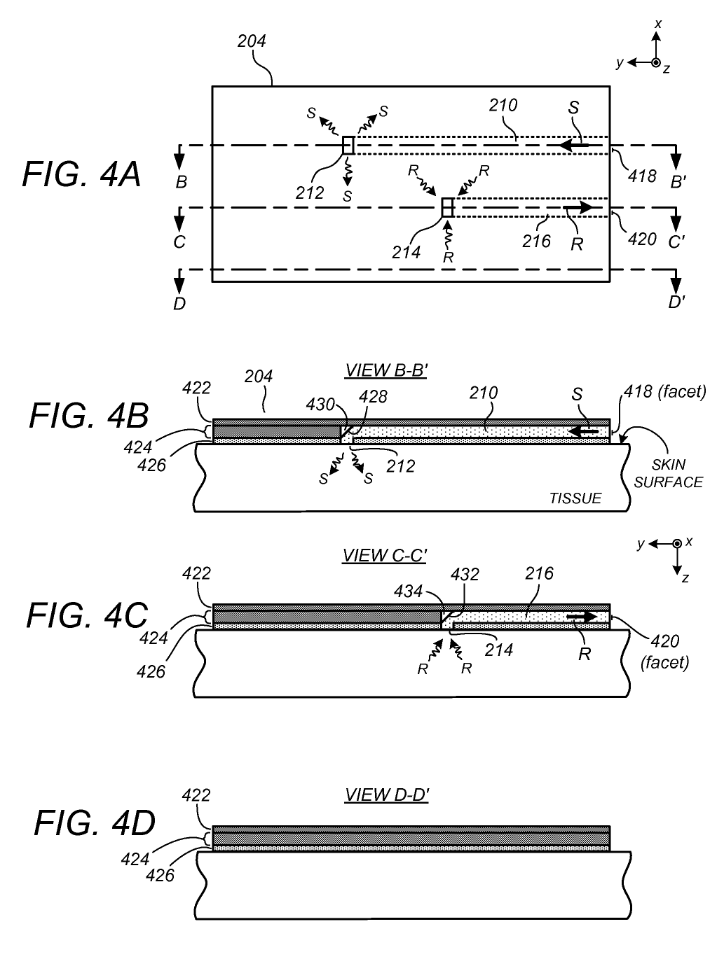

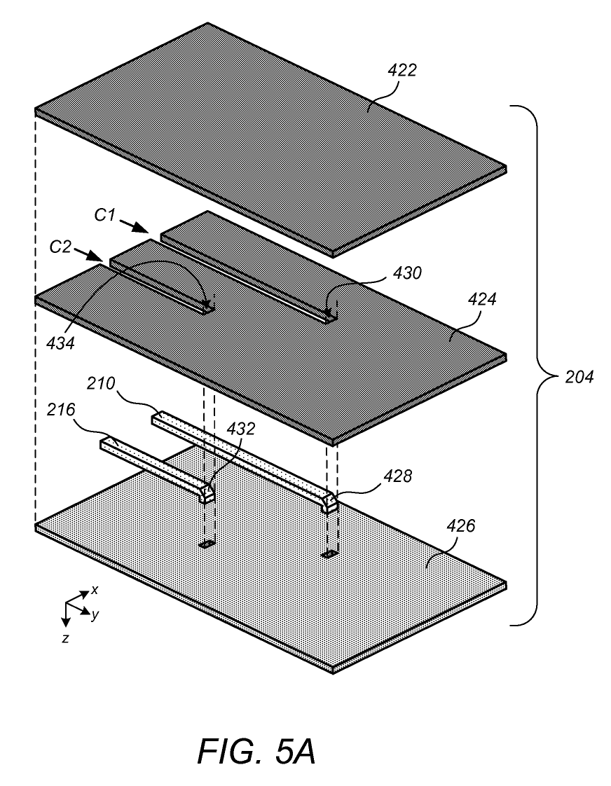

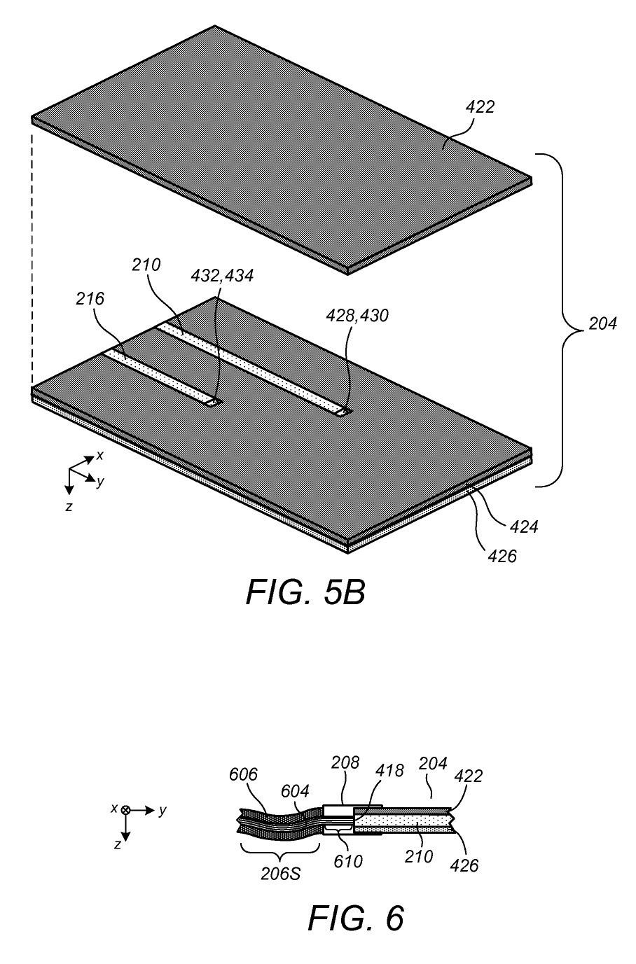

FIG. 4A illustrates a bottom view of the optical coupling patch 204, and FIGS. 4B-4D illustrate side cutaway views of the optical coupling patch 204 along respective cutting planes as positioned along a skin surface. FIGS. 5A-5B illustrate perspective exploded views of the optical coupling patch 204. Optical coupling patch 204 comprises a base layer 426 through which is formed the emitting aperture 212 and the detection aperture 214. The source elastomeric waveguiding member 210 extends laterally across the base layer 426 between a laterally facing end facet 418 and the downwardly facing emitting aperture 212. The detection elastomeric waveguiding member 216 extends laterally across the base layer 426 between a laterally facing end facet 420 and the downwardly facing detection aperture 214. A first cladding layer 424 is formed on the base layer 426 and extends alongside the elastomeric waveguiding members 210/216, and a second cladding layer 422 is formed thereover. Each of the apertures 212 and 214 is laterally distal from its associated end facet 418 and 420, respectively. By laterally distal, it is meant that the optical radiation needs to be laterally guided over a substantial distance relative to the thickness of the optical coupling patch to get from the point of introduction (e.g., the end facet) over to the point of exit (the aperture), consistent with the purpose and form factor of the device. Thus, for example, if the thickness of the optical coupling patch is about 0.15 inches (3.8 mm), then the features and advantages according to the preferred embodiments become especially apparent when the lateral propagation distance of the optical radiation is at least several times that thickness, e.g. at least about 0.60 inches (1.5 cm), although the scope of the preferred embodiments is not so limited.

In accordance with a preferred embodiment, the source elastomeric waveguiding member 210 includes a substantially planar reflecting surface 428 shaped integrally thereinto directly above the emitting aperture 212. The planar reflecting surface 428 can be formed, for example, by virtue of an appropriate mold shape during mold-based formation of the source elastomeric waveguiding member 210, or by using a precision slicing step. Preferably, an air cavity 430 is formed directly adjacent to the planar reflecting surface 428 to facilitate reflection. The reflecting surface 428 is formed at a 45-degree angle relative to the vertical such that source optical radiation that is laterally propagating from the end facet 418 is reflectably redirected in a generally downward direction into the skin surface through the emitting aperture 212. In other preferred embodiments, the angle of the reflecting surface 428, which could alternatively be referred to as a reflective elbow feature, is between about 35 and 55 degrees relative to the vertical. Detection elastomeric waveguiding member 216 is similarly formed with a substantially planar reflecting surface 432 that is molded, sliced, or otherwise fabricated integrally thereinto, whereby radiation that is upwardly emanating at the detection aperture 214 is reflectively redirected to propagate laterally in the detection elastomeric waveguiding member 216 toward the end facet 420. In one preferred embodiment, a reflective coating can be placed on the planar reflective surfaces 428 and 432 for further facilitating the reflective redirection of the optical radiation. In one preferred embodiment, the air gaps 430 and 434 can be filled with a low-index material having a refractive index substantially lower than any of the base layer 426, the cladding layers 422/424, and elastomeric waveguiding members 210/216.

For one exemplary preferred embodiment suitable for spectrophotometric monitoring in the wavelength range of 690 nm-830 nm, the elastomeric waveguiding members 210/216 are formed using a polysiloxane elastomer that exhibits an optical loss of less than 0.3 dB/cm and an index of refraction greater than 1.45 over that wavelength range, while the base layer 426 and cladding layers 422/424 comprise optically opaque polysiloxane elastomers exhibiting indices of refraction less than 1.42 over that wavelength range. For another preferred embodiment, the elastomeric waveguiding members 210/216 are formed using a polysiloxane elastomer that exhibits an optical loss of less than 0.2 dB/cm and a refractive index greater than 1.54 for that wavelength range.

Examples of suitable polysiloxane elastomers for the elastomeric waveguiding members 210/216 are described, for example, in U.S. Pat. No. 7,160,972, which is incorporated by reference herein. Another example of a suitable polysiloxane elastomer for the elastomeric waveguiding members 210/216 is LS-6257 LIGHTSPAN® Optical Thermoset available from NuSil Technology LLC of Carpinteria, Calif., which exhibits a Shore A durometer hardness of 35 (corresponding to a Shore OO durometer hardness of about 83), a refractive index between about 1.55-1.56 for all wavelengths between 690-830 nm, and an optical loss of below 0.2 dB/cm for all wavelengths between 690-830 nm. An example of a suitable polysiloxane elastomer for the base layer 426 and cladding layers 422/424 is NuSil LS-6941 LIGHTSPAN® Optical Thermoset, which exhibits a Shore A durometer hardness of 50 (corresponding to a Shore OO durometer hardness of about 90) and a refractive index between about 1.40-1.41 for all wavelengths between 690-830 nm. Preferably, the NuSil LS-6941 LIGHTSPAN® Optical Thermoset is pigmented with a black pigment for opaqueness, such as MED-4900-2 color masterbatch, also available from NuSil. Another example of a suitable polysiloxane elastomer for the base layer 426 and cladding layers 422/424 is a similarly pigmented version of SILBIONE® RTV 4410 QC A/B Elastomer available from Bluestar Silicones USA Corporation of East Brunswick, N.J., having a Shore A durometer hardness of 10 (corresponding to a Shore OO durometer hardness of about 55). The preferred cladding materials preferably demonstrate adequate biocompatibility and suitability for contact with human skin in accordance with appropriate evaluation standards such as EN/ISO 10993 and appropriate regulatory classifications such as 93/42/CEE European Directive (Class I) or US Pharmocopeia (Class VI).

Fabrication of the optical coupling patch 204 can proceed as follows. The base layer (426) is formed by flowing a thermally curable elastomer into a mold, and then thermally curing the flowed layer. The downward facing apertures (212/214) are formed into the base layer 426 either by virtue of the base layer mold design or by a stamping/cutting process subsequent to base layer cure. Elastomeric waveguiding members (210/216) are then formed upon the base layer (426) either by a molding step or by placing separately prefabricated versions (e.g., separately molded versions) of the elastomeric waveguiding members thereon in appropriate alignment with the apertures. As mentioned previously, the substantially planar reflecting surfaces (428/432) can be formed by virtue of the mold shape (e.g., having appropriately slanted mold sidewalls at those locations), or in a precision post-cure slicing step. The cladding layers (422/424) are then formed atop the base layer/elastomeric waveguiding member assembly in a manner that results in the presence of the air gaps (430/434) next to the planar reflecting surfaces, which can be achieved in a variety of ways. In one example, the first cladding layer (424) is flowed while removable stoppers are positioned over the planar reflecting surfaces (428/432). After curing of the first cladding layer (424), the removable stoppers are removed to expose the air gaps in uncovered form. Finally, a separately prefabricated version (e.g., separately molded version) of the second cladding layer (422) is adhered over the top of the first cladding layer to enclose the air gaps (430/434).

FIG. 6 illustrates a side cut-away view of the optical coupling assembly 202 at an interface between the optical coupling patch 204 and the source fiber optic cable 206S, which are mechanically and optically coupled by the edge adapter 208. The source fiber optic cable 206S comprises an outer sheath 606 and a plurality of optical fibers 604. In one preferred embodiment, the edge adapter 208 is configured with a channel 610 through which the optical fibers 604 are inserted and brought into abutment with the edge facet 418 of the source elastomeric waveguiding member 210. Edge adapter 208 comprises a body made of stainless steel or other rigid material formed into a slot-like shape as shown that compressibly holds the optical coupling patch 204 to maintain the abutment of the edge facet 418 and the optical fibers 604, optionally using an acrylic or epoxy adhesive to further secure the optical coupling patch 204. Optionally, index-matching adhesives or other index-matching methods can be used to reduce reflective losses at the interface between the optical fibers 604 and the source elastomeric waveguiding member 210. Similar interfacing is provided between the return fiber optic cable 206R and the detection elastomeric waveguiding member 216. It is to be appreciated that FIG. 6 represents but one example of a variety of different configurations that can be used to mechanically and optically connect the optical coupling patch 204 with the source/return fiber optic cables 206S/206R as could be achieved by a person skilled in the art without undue experimentation in view of the present disclosure.

For one preferred embodiment, the fiber optic cable assembly 206 and edge adapter 208 can be reusable while the optical coupling patch 204 is disposable, in which case a small, disposable prophylactic (not shown) can be used to cover the edge adapter 208 during each use. In other preferred embodiments, the entire optical coupling assembly 202 including the fiber optic cable assembly 206, the edge adapter 208, and the optical coupling patch 204 are disposable, an option which is made more practical in view of the relatively low material and fabrications cost of the optical coupling patch 204. In still other preferred embodiments, the edge adapter 208 is replaced by a non-rigid, permanent coupling scheme between the fiber optic cable assembly 206 and optical coupling patch 204, the entire optical coupling assembly again being disposable.

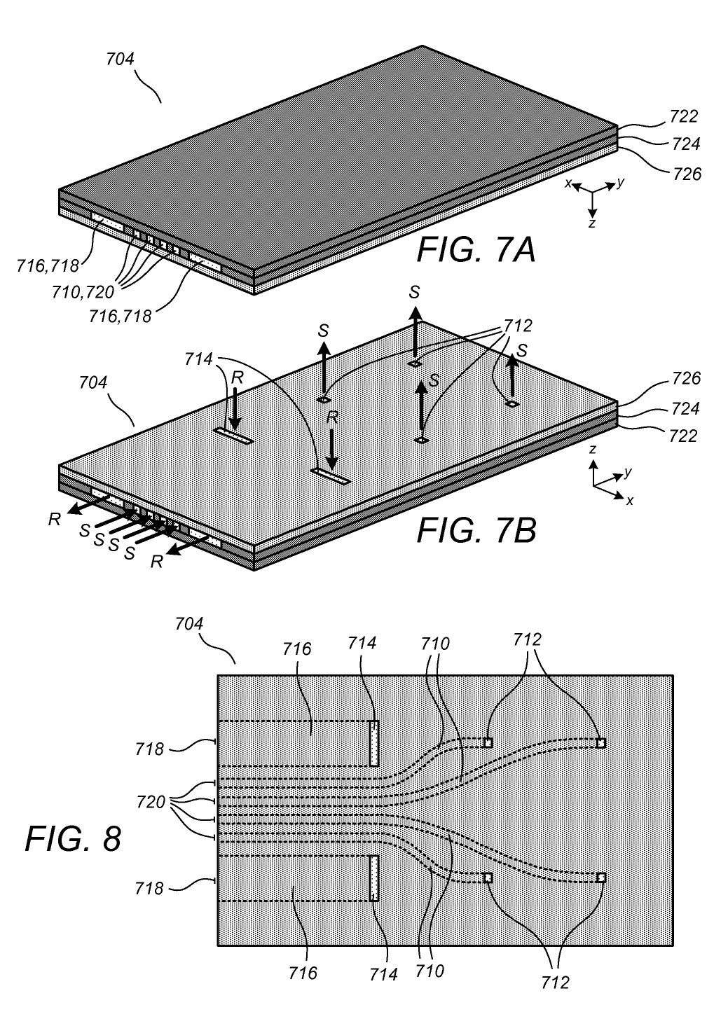

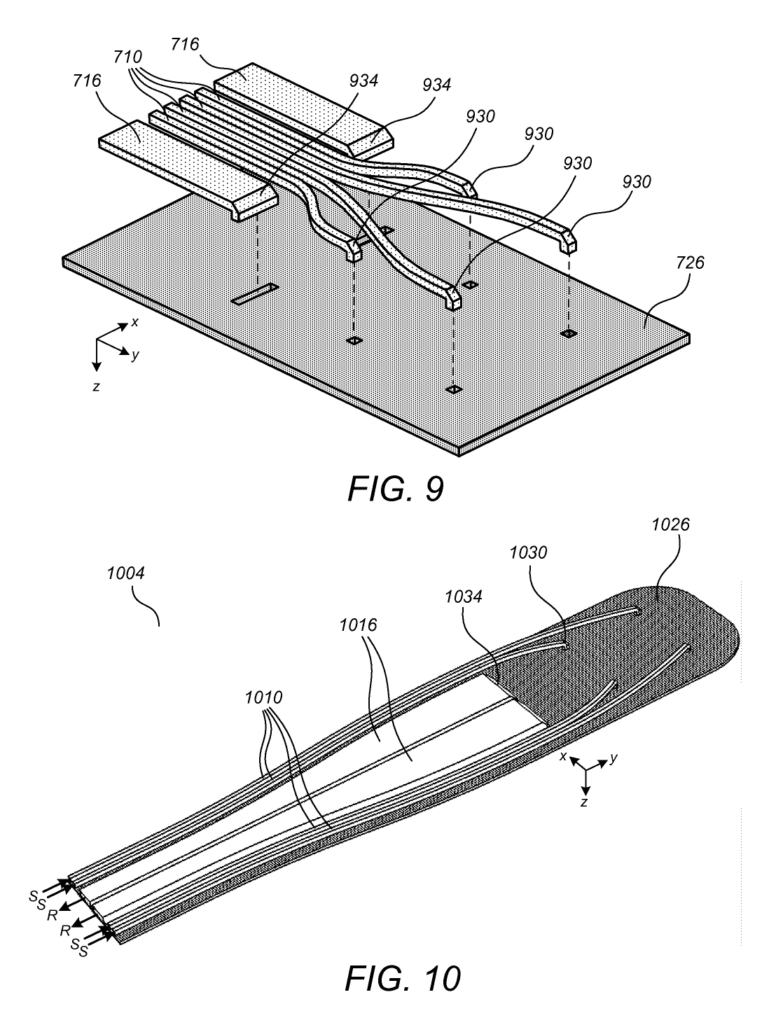

FIGS. 7A-7B illustrate top and bottom perspective views, respectively, of an optical coupling patch 704 that is similar to the optical coupling patch 204 of FIGS. 2-6, supra, except that multiple source and detection elastomeric waveguiding members are provided. The optical coupling patch 704 represents but one of a rich variety of design possibilities for all-elastomeric optical couplers (or virtually all-elastomeric optical couplers, see FIG. 11 and associated description infra) that are within the scope of the preferred embodiments. FIG. 8 illustrates a bottom view, and FIG. 9 illustrates an exploded perspective view, of the optical coupling patch 704. Optical coupling patch 704 includes source elastomeric waveguiding members 710, emitting apertures 714, detecting apertures 716, detection edge facets 718, source edge facets 720, a first cladding layer 724, a second cladding layer 722, and a base layer 726. Source elastomeric waveguiding members 710 each include an angled, substantially planar reflecting surface 930 formed integrally thereinto, and detection elastomeric waveguiding members 716 each include an angled, substantially planar reflecting surface 934 formed integrally thereinto. In one exemplary preferred embodiment, the optical coupling patch 704 has lateral dimensions of about 3 inches (7.62 cm) by 1.5 inches (3.81 cm), and a thickness of about 0.15 inches (3.8 mm). One exemplary size for each of the emitting apertures 714 is about 0.08 inches (2 mm) square, these dimensions also describing the cross-sectional shape of each source elastomeric waveguiding member 710. One exemplary size for each of the detection apertures 716 is about 0.08 inches (2 mm) by 0.24 inches (6 mm), these dimensions also describing the cross-sectional shape of each detection elastomeric waveguiding member 716.

Using the term longitudinal to refer to the general lateral direction between the emitting/detecting apertures 712/714 and the source/detection edge facets 720/718 (i.e., the “y” direction in FIGS. 7A-9), and using the term side-to-side to refer to the lateral direction perpendicular to the longitudinal direction (i.e., the “x” direction in FIGS. 7A-9), the source elastomeric waveguiding members 710 are adiabatically routed in the side-to-side direction as they extend longitudinally between their respective emitting apertures 712 and source edge facets 720, for accommodating a larger cross-sectional size for the detection elastomeric waveguiding members 716. By adiabatically routed, it is meant that any side-to-side routing in the source elastomeric waveguiding members 710 is implemented gradually over a long longitudinal distance as compared to their cross-sectional dimension, for reducing optical loss associated with the side-to-side routing. Because detected photons are precious and few in comparison to source photons in spectrophotometric techniques, it is preferable to make the detection apertures 716 larger in size, rather than the emitting apertures 714 larger in size, in the event such size variation is permitted by the particular spectrophotometric technique being used. For similar reasons, it is preferable that any side-to-side routing that is needed to accommodate the desired device dimensions and aperture patterns be applied to source elastomeric waveguiding members rather than detection elastomeric waveguiding members.

FIG. 10 illustrates a perspective view of an optical coupling patch 1004 according to another preferred embodiment, with cladding layers omitted for clarity of presentation. Shown in FIG. 10 is a base layer 1026 upon which is disposed source elastomeric waveguiding members 1010 including planar reflective surface features 1030 and detection elastomeric waveguiding members 1014 including planar reflective surface features 1034. The detection elastomeric waveguiding members 1016 are adiabatically tapered in a side-to-side cross-sectional dimension and the source elastomeric waveguiding members 1010 are adiabatically routed in the side-to-side direction in order to accommodate a long, slender shape for the optical coupling patch 1004 as may be useful for various monitoring applications.

FIG. 11 illustrates a side cut-away view of an optical coupling patch 1104 according to a preferred embodiment that is similar to the optical coupling patch 204 of FIGS. 2-6, supra, except that one or more rigid reflective optical components is included to facilitate the reflective redirection of the propagating radiation between the lateral and generally vertical directions. Shown in FIG. 11 is a side cut-away view along a detection elastomeric waveguiding member 1116 of the optical coupling patch 1104, which also includes a detection aperture 1114, a base layer 1126, and lower/upper cladding layers 1124/1122, the detection elastomeric waveguiding member 1116 including a substantially planar surface 1132 oriented an angle (e.g., 45 degrees) relative to the vertical. According to the preferred embodiment of FIG. 11, instead of an air gap adjacent to the planar surface 1132, a planar mirror element 1150 is positioned directly adjacent the planar surface 1132 for facilitating the reflective redirection of the optical radiation. In another preferred embodiment shown in FIG. 12, a planar mirror element 1250 is implemented as a silvered (or otherwise reflectively coated) surface of a prism-shaped solid 1251, which could provide for easier manipulation and placement of the planar mirror element in some fabrication scenarios.

Fabrication of the optical coupling patch 1104 can proceed in a manner similar to that of optical coupling patch 204, supra, except that instead of a removable stopper being placed on the planar surface 1132 prior to flowing the lower cladding layer 1124, the planar mirror element 1150 is instead placed there at that time. Also, for the preferred embodiment of FIG. 11, the upper cladding layer 1122 can be formed integrally with the lower cladding layer 1124 in a common flowing and curing step.

FIG. 13 illustrates a side cut-away view of an optical coupling patch 1304 according to a preferred embodiment that is similar in many respects to the optical coupling patch 204 of FIGS. 2-6, supra, except that an internally reflecting prism 1360 is used to deflect the light between vertically and horizontally propagating states. Shown in FIG. 13 is a side cut-away view along a detection elastomeric waveguiding member 1316 of the optical coupling patch 1304, which also includes a detection aperture 1314, a base layer 1326, and lower/upper cladding layers 1324/1322. Here, however, the detection elastomeric waveguiding member 1316 only extends from an end facet 1318 to the prism 1360, rather than having an elbow and extending all the way to the downward-facing detection aperture 1314. The optical radiation is deflected between vertically and horizontally propagating states by the prism 1360.

In the preferred embodiment of FIG. 13, an air gap 1330 is formed adjacent to the internally reflecting surface of the prism 1360 to further facilitate the total internal reflection of the optical radiation. In the preferred embodiment of FIG. 13, an opening 1362 in the base layer 1326 located immediately below the prism 1360 is occupied by air. In the preferred embodiment of FIG. 14 there is no air gap above the prism 1360, but rather the cladding layer 1324 occupies that space. In other preferred embodiments (not shown) a different low-index material can be used to occupy that space. For the preferred embodiment of FIG. 14, total internal reflection can be achieved by using a prism material of sufficiently high index relative to the cladding material for total internal reflection. In the preferred embodiment of FIG. 15 the opening 1362 immediately below the prism 1360 is occupied by the same elastomeric material as the detection elastomeric waveguiding member 1316. In other preferred embodiments (not shown) different materials, such as index-matching materials, can be used to occupy the opening 1362.

Whereas many alterations and modifications of the preferred embodiments will no doubt become apparent to a person of ordinary skill in the art after having read the foregoing description, it is to be understood that the particular embodiments shown and described by way of illustration are in no way intended to be considered limiting. By way of example, although the optical coupling patches according to one or more of the preferred embodiments described supra are bidirectional in function (i.e., providing both optical source coupling and optical detection coupling functions), optical coupling patches that are unidirectional in function (i.e., providing only optical source coupling, or only optical detection coupling) are also within the scope of the preferred embodiments.

By way of further example, although the source radiation (detected radiation) is illustrated in one or more of the preferred embodiments supra as entering (exiting) the optical coupling patch at a laterally facing end facet, in alternative preferred embodiments the source radiation (detected radiation) may enter (exit) the optical coupling patch along a vertically facing facet. In such cases, the optical radiation would be deflected near the entry facet (exit facet) between vertically and horizontally propagating states using a deflection scheme similar to one or more of the above-described deflection schemes (for example, the deflection scheme near apertures 212/214 of FIG. 2, supra). Thus, in such cases, the optical radiation would be deflected twice inside the optical coupling patch, at respective locations that are laterally distal from each other, with the optical radiation being laterally guided between those locations by an elastomeric waveguiding member. Thus, reference to the details of the described embodiments are not intended to limit their scope, which is limited only by the scope of the claims set forth below.

What is claimed is:

1. An optical coupling patch for use in spectrophotometric patient monitoring, comprising:
a flexible base layer having a skin-contacting surface and a first aperture formed therethrough, said flexible base layer comprising a first elastomeric material having a first refractive index, said first aperture establishing an optical interface with a skin surface when said flexible base layer is placed against said skin surface;
an elastomeric waveguiding member laterally disposed on a surface of said flexible base layer opposite said skin-contacting surface, said elastomeric waveguiding member comprising a second elastomeric material having a second refractive index greater than said first refractive index and being configured to guide optical radiation between (i) a laterally propagating state at a first location laterally distal from said first aperture, and (ii) a generally vertically propagating state at said first aperture, wherein said elastomeric waveguiding member includes a substantially planar reflecting surface shaped integrally thereinto near said first aperture, said reflecting surface being oriented at an angle that causes reflective redirection of the optical radiation between said laterally propagating state and said vertically propagating state; and
a flexible cladding material having a third refractive index less than said second refractive index and selectively covering said elastomeric waveguiding member such that a cavity occupied by one of air and a low-index material having a fourth refractive index less than said third refractive index is formed directly adjacent said reflecting surface for facilitating said reflective redirection of the optical radiation.

2. The optical coupling patch of claim 1, said elastomeric waveguiding member having a laterally facing end facet at said first location, said elastomeric waveguiding member thereby guiding the optical radiation between said end facet and said first aperture.

3. The optical coupling patch of claim 2, said laterally facing end facet being adapted for coupling to an optical radiation source external to said optical coupling patch, said elastomeric waveguiding member guiding the source optical radiation to said first aperture, the source radiation thereby propagating into tissue underlying the skin surface.

4. The optical coupling patch of claim 3, said laterally facing end facet being a first end facet and said elastomeric waveguiding member being a first elastomeric waveguiding member, said optical coupling patch further comprising a second elastomeric waveguiding member formed similarly to said first elastomeric waveguiding member and extending between a second laterally facing end facet and a second aperture formed through said skin-contacting surface, said second laterally facing end facet being adapted for coupling to an optical radiation detector external to said optical coupling patch, said second elastomeric waveguiding member guiding optical radiation received through said second aperture from the skin surface to said second laterally facing end facet for detection by said optical radiation detector.

5. The optical coupling patch of claim 2, said laterally facing end facet being adapted for coupling to an optical radiation detector external to said optical coupling patch, said elastomeric waveguiding member guiding optical radiation received through said first aperture from the skin surface to said laterally facing end facet for detection by said optical radiation detector.

6. The optical coupling patch of claim 5, said laterally facing end facet being a first end facet and said elastomeric waveguiding member being a first elastomeric waveguiding member, said optical coupling patch further comprising a second elastomeric waveguiding member formed similarly to said first elastomeric waveguiding member and extending between a second laterally facing end facet and a second aperture formed through said skin-contacting surface, said second laterally facing end facet being adapted for coupling to an optical radiation source external to said optical coupling patch, said second elastomeric waveguiding member guiding the source optical radiation to said second aperture, the source radiation thereby propagating into tissue underlying the skin surface.

7. The optical coupling patch of claim 1, wherein said flexible base layer, said elastomeric waveguiding member, and said flexible cladding material each comprise a curable polysiloxane elastomer having a Shore OO durometer hardness between about 25 and 95.

8. The optical coupling patch of claim 7, wherein for an optical radiation wavelength range of about 690 nm-830 nm, said elastomeric waveguiding member exhibits an optical loss of less than 0.3 dB/cm and an index of refraction greater than 1.45, and wherein said flexible base layer and said flexible cladding material exhibit an index of refraction of less than 1.42 for said wavelength range.

9. The optical coupling patch of claim 8, wherein said elastomeric waveguiding member exhibits an optical loss of less than 0.2 dB/cm and a refractive index greater than 1.54 for said wavelength range.

10. The optical coupling patch of claim 1, wherein said angle of said reflecting surface is between about 35 and 55 degrees relative to said first aperture.

11. The optical coupling patch of claim 1, further comprising a reflective coating disposed on said elastomeric waveguiding member at said substantially planar reflecting surface for further facilitating said reflective redirection of the optical radiation.

12. A flexible, slab-like optical coupling patch for use in spectrophotometric patient monitoring, the optical coupling patch having a bottom surface for contacting a skin surface of a patient and a side edge including first and second end facets, the optical coupling patch being operable to guide source radiation received at the first end facet to a downward facing first aperture formed in the bottom surface for downward introduction into the patient, the coupling patch being further operable to receive radiation emanating upwardly from the patient at a second aperture formed in the bottom surface and to guide the received radiation from the second aperture to the second end facet, the optical coupling patch comprising:
a flexible base layer including said bottom surface;
first and second elastomeric waveguiding members disposed on said base layer and extending from said first and second end facets, respectively, to said first and second apertures, respectively;
a flexible first cladding layer disposed on said base layer and extending alongside said first and second elastomeric waveguiding members; and
a flexible second cladding layer disposed atop said first cladding layer and said first and second elastomeric waveguiding members, wherein said base layer, said first cladding layer, and said second cladding layer each have an index of refraction less than that of either of said first and second elastomeric waveguiding members;
wherein each said first and second elastomeric waveguiding members includes a substantially planar surface shaped integrally thereinto near its respective aperture that is oriented at an angle between about 35 and 55 degrees relative thereto, whereby the source radiation propagating laterally in said first elastomeric waveguiding member is reflectively redirected downward toward said first aperture, and whereby the upwardly emanating radiation received at said second aperture is reflectively redirected to propagate laterally in said second elastomeric waveguiding member toward the second end facet.

13. The optical coupling patch of claim 12, each said first and second elastomeric waveguiding member being generally rectangular in cross-section and each having a lower surface, two sidewall surfaces, and a top surface extending therealong, wherein said flexible first and second cladding layers are formed integrally with each other into a common cladding layer that covers said sidewall and top surfaces of each of said first and second elastomeric waveguiding members.

14. The optical coupling patch of claim 12, wherein said base layer, said first and second elastomeric waveguiding members, and said flexible first and second cladding layers each comprise a curable polysiloxane elastomer material having a Shore OO durometer hardness between about 25 and 95.

15. The optical coupling patch of claim 12, wherein for an optical radiation wavelength range of about 690 nm-830 nm, said first and second elastomeric waveguiding members each exhibit an optical loss of less than 0.3 dB/cm and an index of refraction greater than 1.45, and wherein said base layer and said flexible first and second cladding layers each exhibit an index of refraction of less than 1.42 for said wavelength range.

16. The optical coupling patch of claim 15, wherein said first and second elastomeric waveguiding members each exhibit an optical loss of less than 0.2 dB/cm and a refractive index greater than 1.54 for said wavelength range.

17. The optical coupling patch of claim 12, wherein said flexible first and second cladding layers are configured such that, for each of said first and second elastomeric waveguiding members, an air cavity is formed along the integrally shaped, substantially planar surface thereof for further facilitating said reflective redirection of the optical radiation.

18. The optical coupling patch of claim 12, further comprising a reflective coating disposed on said substantially planar surface of each of said first and second elastomeric waveguiding members for further facilitating said reflective redirection of the optical radiation.

19. The optical coupling patch of claim 12, further comprising, for each of said first and second elastomeric waveguiding members, a reflective prism disposed along said substantially planar surface for further facilitating said reflective redirection of the optical radiation.

20. The optical coupling patch of claim 12, further comprising, for each of said first and second elastomeric waveguiding members, a planar mirror disposed along said substantially planar surface for further facilitating said reflective redirection of the optical radiation.

21. A method for fabricating a flexible, slab-like optical coupling patch for use in spectrophotometric patient monitoring, the optical coupling patch having a bottom surface for contacting a skin surface of a patient and a side edge, comprising:
providing a flexible base layer comprising an elastomeric material, the base layer extending to the side edge and having a lower surface corresponding to the bottom surface of the optical coupling patch and an upper surface opposite said lower surface, the base layer having an opening extending through said lower and upper surfaces;
forming an elastomeric waveguiding member on the upper surface of the base layer extending laterally thereacross between the side edge and the opening, wherein said elastomeric waveguiding member comprises:
a first end facet facing in a lateral direction at said side edge;
a second end facet facing downwardly into said first opening; and
a substantially planar surface integrally formed into the first elastomeric waveguiding member by virtue of its outer shape at a location directly above said second end facet, said substantially planar surface being oriented at an angle between about 35 and 55 degrees relative to said second end facet; and
forming at least one flexible cladding layer that covers said base layer and said elastomeric waveguiding member.

22. The method of claim 21, wherein said base layer, said elastomeric waveguiding member, and said at least one flexible cladding layer each comprise a curable polysiloxane elastomer material having a Shore OO durometer hardness between about 25 and 95.

23. The method of claim 21, wherein said elastomeric waveguiding member extends downwardly into said opening such that said second end facet is substantially flush with said bottom surface.

24. The method of claim 21, wherein said forming the elastomeric waveguiding member upon the upper surface of the base layer comprises flowing a curable optical elastomer into a mold above said base layer, said mold defining the outer shape of said elastomeric waveguiding member including said angularly oriented, substantially planar surface.

25. The method of claim 21, wherein said forming the elastomeric waveguiding member upon the upper surface of the base layer comprises:
flowing a curable optical elastomer into a mold above said base layer, said mold defining the outer shape of said elastomeric waveguiding member not including said angularly oriented, substantially planar surface; and
subsequent to curing of the optical elastomer, mechanically slicing said elastomeric waveguiding member at said location directly above said second end facet to form said angularly oriented, substantially planar surface.

26. The method of claim 21, wherein said forming the elastomeric waveguiding member upon the upper surface of the base layer comprises:
receiving a prefabricated version of the elastomeric waveguiding member including said angularly oriented, substantially planar surface and said second end facet; and
placing said prefabricated version onto said base layer such that said second end facet is positioned directly into or directly above said opening; and
adhering said prefabricated version to said base layer.

27. The method of claim 21, wherein said forming at least one flexible cladding layer comprises forming an air cavity directly adjacent said angularly oriented, substantially planar surface such that said air cavity and said elastomeric waveguiding member are collectively enveloped by said at least one flexible cladding layer.

28. The method of claim 27, wherein said forming the air cavity directly adjacent said angularly oriented, substantially planar surface comprises:
placing a removable stopper directly over said angularly oriented, substantially planar surface of said elastomeric waveguiding member;
flowing a curable cladding elastomer onto said base layer around said elastomeric waveguiding member and said removable stopper to a predetermined height corresponding to a desired upper level of the air cavity to thereby form a first cladding layer;
subsequent to curing of the first cladding layer, removing the removable stopper to thereby create said air cavity in uncovered form; and
covering said first cladding layer and said air cavity with a prefabricated version of a second cladding layer, thereby enclosing said air cavity.

29. The method of claim 21, further comprising applying a reflective coating to said angularly oriented, substantially planar surface of said of said elastomeric waveguiding member.

30. The method of claim 21, further comprising positioning a planar mirror element directly against said angularly oriented, substantially planar surface of said elastomeric waveguiding member, wherein said at least one flexible cladding layer further covers said planar mirror element.

31. The method of claim 21, wherein for an optical radiation wavelength range of about 690 nm-830 nm, said elastomeric waveguiding member exhibits an optical loss of less than 0.3 dB/cm and an index of refraction greater than 1.45, and wherein said base layer and at least one cladding layer each exhibit an index of refraction of less than 1.42 for said wavelength range.

32. The method of claim 31, wherein said elastomeric waveguiding member each exhibits an optical loss of less than 0.2 dB/cm and a refractive index greater than 1.54 for said wavelength range.

33. The method of claim 21, wherein each said providing the flexible base layer, forming the elastomeric waveguiding member, and said forming at least one flexible cladding layer comprises flowing a thermally curable polysiloxane elastomer into a mold and curing the resultant formed layer.

34. A flexible, slab-like optical coupling patch for use in spectrophotometric patient monitoring, the optical coupling patch having a bottom surface for contacting a skin surface of a patient and a downward facing first aperture formed in the bottom surface, the optical coupling patch being operable to guide optical radiation between (i) a laterally propagating state at a first location laterally distal from said first aperture, and (ii) a generally vertically propagating state at the first aperture, the optical coupling patch comprising:
a flexible base layer including said skin-contacting surface and a first opening formed therethrough that establishes said first aperture, said first aperture establishing an optical interface with the skin surface when said flexible base layer is placed thereagainst;
a light deflecting member disposed above said first opening and configured to deflect optical radiation between a generally vertically propagating state thereat and a laterally propagating state thereat; and
an elastomeric waveguiding member disposed on a surface of said flexible base layer opposite said skin-contacting surface and extending laterally across the optical coupling patch from said light deflecting member to said first location laterally distal from said first aperture.

35. The optical coupling patch of claim 34, wherein said light deflecting member comprises a prism configured and positioned relative to said elastomeric waveguiding member and said first aperture such that the prism deflects the optical radiation by total internal reflection.

36. The optical coupling patch of claim 35, further comprising a flexible cladding material selectively covering said elastomeric waveguiding member and said prism such that an air cavity is formed directly adjacent to a light deflecting surface of the prism to facilitate the total internal reflection of the optical radiation.

37. The optical coupling patch of claim 36, said elastomeric waveguiding member having a laterally facing end facet at said first location, said optical coupling patch thereby guiding the optical radiation between said end facet and said first aperture.

38. The optical coupling patch of claim 37, said laterally facing end facet being adapted for coupling to an optical radiation source external to said optical coupling patch, said optical coupling patch guiding the source optical radiation to said first aperture, the source radiation thereby propagating into tissue underlying the skin surface.

39. The optical coupling patch of claim 38, said laterally facing end facet being a first end facet, said elastomeric waveguiding member being a first elastomeric waveguiding member, said prism being a first prism, further comprising:
a second elastomeric waveguiding member, a second prism, and a second opening formed through the flexible base layer to establish a second aperture;
wherein said second elastomeric waveguiding member, said second opening, said second prism, and said second aperture are formed similarly to said first elastomeric waveguiding member, said first elastomeric waveguiding member, said first opening, and said first prism and extend between a second laterally facing end facet and said second aperture to guide optical radiation received through said second aperture from the skin surface to said second laterally facing end facet for detection by an external optical radiation detector.

40. The optical coupling patch of claim 36, wherein said flexible base layer, said elastomeric waveguiding member, and said flexible cladding material each comprise a curable polysiloxane elastomer having a Shore OO durometer hardness between about 25 and 95.

41. The optical coupling patch of claim 40, wherein for an optical radiation wavelength range of about 690 nm-830 nm, said elastomeric waveguiding member exhibits an optical loss of less than 0.3 dB/cm and an index of refraction greater than 1.45, and wherein said flexible base layer and said flexible cladding material exhibit an index of refraction of less than 1.42 for said wavelength range.

42. The optical coupling patch of claim 41, wherein said elastomeric waveguiding member exhibits an optical loss of less than 0.2 dB/cm and a refractive index greater than 1.54 for said wavelength range.
